(19) United States
(12) Patent Application Publication
(10) Pub. No.: US 2023/0243744 A1
(43) Pub. Date: Aug. 3, 2023

(54) METHOD AND SYSTEM FOR AUTOMATICALLY DETECTING AND RECONSTRUCTING SPECTRUM PEAKS IN NEAR INFRARED SPECTRUM ANALYSIS OF TEA

(71) Applicant: Anhui University, Hefei (CN)

(72) Inventors: Tianhong PAN, Hefei (CN); Chao WU, Hefei (CN); Qi CHEN, Hefei (CN); Shan CHEN, Hefei (CN); Yuan FAN, Hefei (CN); Qiong WU, Hefei (CN); Xiaofeng YU, Hefei (CN)

(21) Appl. No.: 18/192,733

(22) Filed: Mar. 30, 2023

Related U.S. Application Data
(63) Continuation of application No. PCT/CN2021/138605, filed on Dec. 16, 2021.

(30) Foreign Application Priority Data
Dec. 14, 2021 (CN) 202111526109.5

(51) Int. Cl. G01N 21/359; G01N 21/3577; G01N 33/14

(52) U.S. Cl. CPC G01N 21/359; G01N 21/3577; G01N 33/14

(57) ABSTRACT

Disclosed are a method and a system for automatically detecting and reconstructing spectrum peaks in near infrared spectrum analysis of tea, including the following steps: firstly collecting initial spectrum data, then initializing parameters, then calculating the position and width of absorption peaks, then updating correlation coefficients and screening sparse blocks, then calculating the cost function and the expectation, then determining termination conditions, and finally outputting reconstruction data.

### CROSS-REFERENCE TO RELATED APPLICATIONS

This application is a continuation of PCT/CN 2021/138605, filed on Dec. 16, 2021 and claims priority of Chinese Patent Application No. 202111526109.5, filed on Dec. 14, 2021, the entire contents of which are incorporated herein by reference.

### TECHNICAL FIELD

The application relates to that technical field of spectrum analysis, and in particular to a method and a system for automatically detecting and reconstructing spectrum peaks in near infrared spectrum analysis of tea.

### BACKGROUND

At present, tea is one of the most important drinks in China, and the classification of tea quality is related to many indexes such as tea polyphenols, caffeine, amino acids, sugar content, etc. For a long time, people's mastery of the quality of each process in tea processing mainly depends on sensory evaluation, lacking quantitative processing evaluation standards, and the quality of processed products is mainly judged by sensory evaluation, lacking a digital rapid evaluation method that takes into account the main chemical components and external morphological characteristics. With the continuous development of China's import and export trade and the continuous improvement of people's material needs, more detailed analysis requirements are put forward for industrial classification and quality identification, and the existing experience-based identification methods are no longer suitable for large-scale and high-precision analysis requirements.

As an important branch of spectroscopy, near infrared spectroscopy has the characteristics of a pollution-free, low costs and short detection period, and has been widely used in the analysis of tea-related industries. However, in the actual analysis process, due to the high dimension, collinearity and limited number of spectrum peaks of spectrum data, there are a large number of irrelevant characteristic variables in the collected spectrum data in the full wavelength range; according to Beer-Lambert law, the performance of the analytical model depends on the validity of the modeling data, and the presence of irrelevant variables will destroy the data characteristics, so it is necessary to carry out necessary characteristic selection operations on the spectrum data before quantitative analysis.

At present, the widely used sparse reconstruction methods mainly include Group Lasso, Block Orthogonal Matching Pursuit, Sparse Bayesian Learning and Block Sparse Bayesian Learning. The sparse reconstruction method based on Block Sparse Bayesian Learning is the most promising. The sparse reconstruction method based on Block Sparse Bayesian Learning may effectively avoid solving the norm problem and introduce the block structure, but the randomness of the location and size of the block is not considered, and the block is divided without accurately determining the spectrum peak position, which easily leads to the phenomenon that the characteristic variables of the spectrum peak position are sparse, and consequently inaccurate reconstruction results. Aiming at the near infrared spectrum data of tea with multiple overlapping peaks, how to accurately select the characteristic variables of the spectrum peaks is very important for the process of tea qualitative and quantitative analysis. Therefore, the application provides a method and a system for automatically detecting and reconstructing spectrum peaks in near infrared spectrum analysis of tea to solve the problems existing in the prior art.

### SUMMARY

In view of the above problems, the objective of the present application is to propose a method and a system for automatically detecting and reconstructing spectrum peaks in near infrared spectrum analysis of tea. In this method, the number and spectrum peak position of near-infrared spectrum may be accurately judged by using a Block Sparse Bayesian Learning method for automatically detecting the spectrum peak position and determining the peak width, so that the spectrum peak characteristics may be accurately reconstructed and selected, avoiding the problems of erroneous reconstruction and loss in the reconstruction process of algorithm, and the selection of absorption peak characteristics of near-infrared spectrum data with multiple overlapping peaks may be realized based on the strategy of sparse reconstruction and automatic detection of spectrum peaks.

In order to achieve the objective of the application, the application is realized by the following technical scheme: a method for automatically detecting and reconstructing spectrum peaks in near infrared spectrum analysis of tea, including the following steps:

S1, firstly, collecting tea samples to be detected, then obtaining near infrared spectrum data of the tea samples, and forming initial data;

S2, firstly, obtaining the initial data, and then initializing parameters of Block Sparse Bayesian Learning method including correlation coefficient γ, iteration times T, noise variance λ, symmetric positive semidefinite matrix A and relative error of correlation coefficient;

S3, according to spectrum characteristics in the initial data, calculating an absorption peak position in the spectrum based on a first-order deviation and a second-order deviation;

S4, calculating a spectrum peak width based on a half-peak height according to the calculated absorption peak position;

S5, calculating the symmetric positive semidefinite matrix, a correlation structure matrix and the correlation coefficient of each block according to a sparsity control coefficient of each block;

S6, calculating an error value of each block in the initial data based on a cost function, and screening sparse blocks;

S7, calculating an expectation and a variance of a spectrum posterior probability;

S8, solving superparameters by using a minimization cost function, and updating the noise variance λ in initialization parameters;

S9, calculating the relative error of the block correlation coefficient and the current iteration times; if the relative error is less than the set error coefficient 11 or the current iteration times are larger than the set iteration times T, then turning to S10, otherwise turning to the S5; and

S10, determining a final tea sparse reconstruction data and output the data by using the spectrum posterior probability expectation.

As a further improvement, in the S2, an optimization function of the Block Sparse Bayesian Learning method is as follows:

L=log|λI+ΩΣ₀Ω^{T}|+y^{T}(λI+ΩΣ₀Ω^{T})⁻¹y,

where I represents an identity matrix, y represents a compressed matrix of spectrum obtained by measuring matrix Ω, Ω∈ [symbol] ^{M×N} is a measuring matrix, and Σ₀∈ [symbol] ^{N×N} is a variance matrix of all blocks, which is expressed as:

Σ₀=diag{γ₁B₁, . . . ,γᵢBᵢ, . . . ,γ_{g}B_{g}},

where γᵢ represents a block correlation coefficient of an i-th block, and Bᵢ represents a structure matrix of the i-th block;

As a further improvement, in the S3, a spectrum peak position is determined and calculated as follows:

Δxⱼ=xⱼ−xⱼ₋₁

Δ²xⱼ=Δxⱼ−Δxⱼ₋₁

s.t. Δxⱼ=0 and Δ²xⱼ<0

where Δxⱼ and Δ²xⱼ are the first-order deviation and the second-order deviation of a spectrum peak point xⱼ, respectively.

As a further improvement, in the S4, the spectrum peak width is calculated as expressed as follows:

$$\{ \begin{matrix}w = n - m , n > m \\ s . t . {x}_{n} = {x}_{m} = \frac{1}{2} H\end{matrix} ,$$

where n and m are indexes xₙ and xₘ, respectively, and a relative height difference H is expressed as follows:

$$\{ \begin{matrix}H = \vert {x}_{j} - {x}_{i} \vert , {x}_{i} > {x}_{k} \\ H = \vert {x}_{j} - {x}_{k} \vert , {x}_{i} < {x}_{k}\end{matrix} ,$$

where xᵢ and xₖ are a starting point and an ending point of the spectrum peak, respectively.

As a further improvement, in the S5, the symmetric positive semidefinite matrix, the correlation structure matrix and the correlation coefficient are expressed as follows:

$${A}_{i} = {s}_{i}^{- 1} ( {q}_{i} {q}_{i}^{T} - {s}_{i} ) {s}_{i}^{- 1}$$

$${γ}_{i} {=}^{△} \frac{1}{{d}_{i}} Tr ( {A}_{i} )$$

$${B}_{i} = \frac{{A}_{i}}{{γ}_{i}} ,$$

where sᵢ=Ωᵢ^{T}D₋ᵢ⁻¹Ωᵢ, qᵢ=Ωᵢ^{T}D₋ᵢ⁻¹y and D₋ᵢ=λI+Σ_{m=1,m≠1}^{g}ΩₘγₘBₘΩₘ^{T}, dᵢ represents a size of the i-th block.

As a further improvement, in the S6, an error of the cost function is calculated as follows:

L=log|λI+ΩΣ₀Ω^{T}|+y^{T}(λI+ΩΣ₀Ω^{T})⁻¹y,

where Ω∈ [symbol] ^{M×N} is the measuring matrix and Σ₀∈ [symbol] ^{N×N} is the variance matrix of all the blocks, which is expressed as follows:

ΔL(i)=L(Aᵢ^{(t)})−L(Aᵢ^{(t-1)}),

where Aᵢ^{(t)} represents the variance matrix of the i-th block in an iteration of t-th step.

As a further improvement, in the S7, the posterior probability expectation is calculated as follows:

μ_{X}=Σ₀Ω^{T}(λI+ΩΣ₀Ω^{T})⁻¹y,

where y is the compressed matrix of the spectrum obtained by measuring the matrix Ω.

As a further improvement, in the S9, relative error judgment conditions are expressed as follows:

$$\frac{‖ {γ}^{( t )} - {γ}^{( t - 1 )} ‖}{‖ {γ}^{( t - 1 )} ‖} \leq η ,$$

where γ^{(t)} is the correlation coefficient of the t-th iteration.

A system for automatically detecting and reconstructing spectrum peaks in near infrared spectrum analysis of tea, including:

a sample acquisition module is used for obtaining the tea samples, obtaining tea near infrared spectrum data and forming the initial data;

a parameter initialization module is used for obtaining the initial data and initializing the parameters of Block Sparse Bayesian Learning method, where the initialization parameter include correlation coefficient γ, iteration times T, noise variance λ, symmetric positive semidefinite matrix A and relative error η of correlation coefficient;

a spectrum peak position calculation module is used for determining the absorption peak position according to the first-order deviation and the second-order deviation of the spectrum data;

a spectrum peak width calculation module is used for determining the peak width according to the half-peak height of the absorption peak;

a correlation coefficient calculation module is used for calculating the sparsity control coefficient of each block to obtain the correlation coefficient;

a screening module is used for calculating the error value of each block and screening the sparse blocks according to the cost function;

an expectation and variance calculation module is used for obtaining the expectation and the variance according to a posterior probability distribution of the spectrum;

a noise variance update module is used for solving the superparameters according to the minimization cost function to obtain noise variance update;

a judging module is used for calculating the relative error of the block correlation coefficient and the current iteration times, and if the relative error is less than the set error coefficient η or the current iteration times are greater than the set iteration times T, the judging is stopped; otherwise, the calculating block correlation coefficient and a block screening module are called again to calculate the sparse reconstruction;

a data correction module is used for determining and outputting the final tea sparse reconstruction data by using the spectrum posterior probability expectation.

The method has the following beneficial effects.

In this method, the number and spectrum peak position of near-infrared spectrum may be accurately judged by using a Block Sparse Bayesian Learning method for automatically detecting the spectrum peak position and determining the peak width, so that the spectrum peak characteristics may be accurately reconstructed and selected, avoiding the problems of erroneous reconstruction and loss in the reconstruction process of algorithm, and the selection of absorption peak characteristics of near-infrared spectrum data with multiple overlapping peaks may be realized based on the strategy of sparse reconstruction and automatic detection of spectrum peaks, so that the automatic detection and reconstruction with high-precision of the absorption peaks of the near infrared spectrum data of green tea may be realized, which is beneficial to improving the accuracy of tea detecting grade and expanding market trade.

### BRIEF DESCRIPTION OF THE DRAWINGS

In order to more clearly explain the embodiments of the present application or the technical scheme in the prior art, the drawings needed in the description of the embodiments or the prior art will be briefly introduced below. Obviously, the drawings in the following description are only some embodiments of the present invention, and other drawings may be obtained according to these drawings without creative labor for ordinary people in the field.

FIG. 1 is a schematic flow diagram of a method according to Embodiment 1 of the present application.

FIG. 2 is a schematic diagram of tea spectrum data according to Embodiment 1 of the present application.

FIG. 3 is a schematic diagram of the determination and reconstruction results of leaf absorption peaks according to Embodiment 1 of the present application.

FIG. 4 is a comparative schematic diagram of analysis results of different reconstruction methods according to Embodiment 1 of the present application.

FIG. 5 is a schematic diagram of the system structure of Embodiment 2 of the present application.

### DETAILED DESCRIPTION OF THE EMBODIMENTS

In the following, the technical scheme in the embodiment of the application will be clearly and completely described with reference to the attached drawings. Obviously, the described embodiment is only a part of the embodiment of the application, but not the whole embodiment. Based on the embodiments in the present application, all other embodiments obtained by ordinary technicians in the field without creative work belong to the scope of protection of the present application.

### Embodiment 1

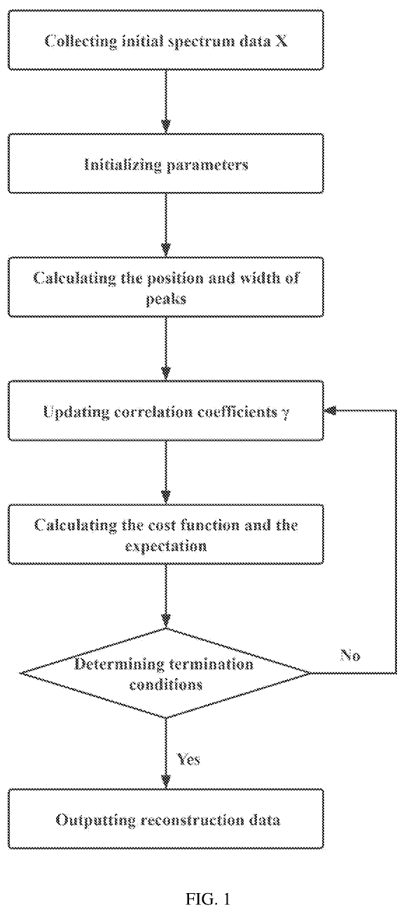

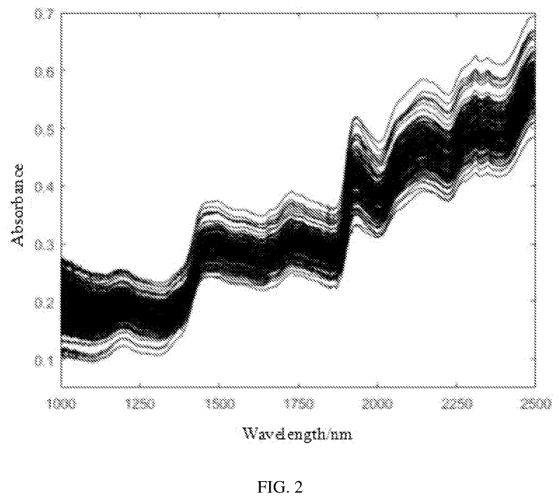

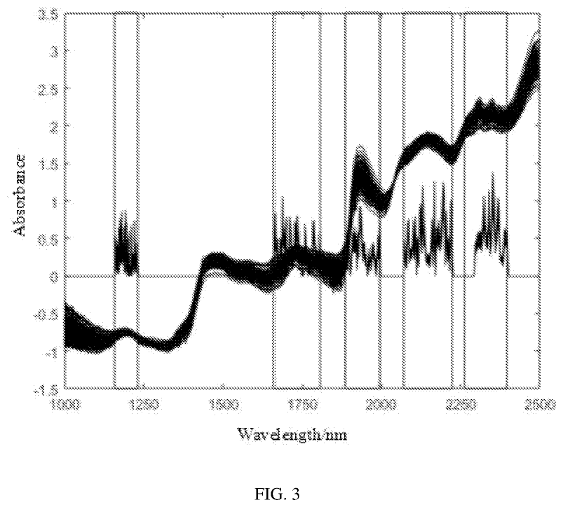

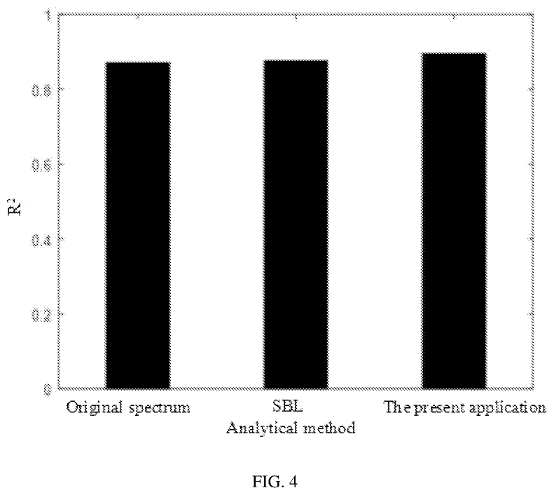

Referring to FIG. 1, FIG. 2, FIG. 3 and FIG. 4, this embodiment provides a method for automatically detecting and reconstructing spectrum peaks in near infrared spectrum analysis of tea, including the following steps.

Step 1: firstly, collecting tea samples to be detected, then obtaining near infrared spectrum data of the tea samples and forming initial data, and the collected green tea spectrum data is X∈ [symbol] ^{158×830}, and the sugar content data is Y∈ [symbol] ^{158×1}.

S2, firstly, obtaining the initial data, and then initializing parameters of Block Sparse Bayesian Learning method including correlation coefficient γ, iteration times T, noise variance λ, symmetric positive semidefinite matrix A and relative error η of correlation coefficient; the initialization parameters are set as follows: iteration times T=100, noise variance λ=10-2, correlation coefficient γ=0, relative error η=10⁻⁸ and measuring matrix Ω=rand( );

The optimization function of the Block Sparse Bayesian Learning method is as follows:

L=log|λI+ΩΣ₀Ω^{T}|+y^{T}(λI+ΩΣ₀Ω^{T})⁻¹y,

where I represents an identity matrix, y represents a compressed matrix of spectrum obtained by measuring matrix Ω, Ω∈ [symbol] ^{M×N} is a measuring matrix, and Σ₀∈ [symbol] ^{N×N} is a variance matrix of all blocks, which is expressed as:

Σ₀=diag{γ₁B₁, . . . ,γᵢBᵢ, . . . ,γ_{g}B_{g}},

where γᵢ represents a block correlation coefficient of an i-th block, and Bᵢ represents a structure matrix of the i-th block.

S3, according to spectrum characteristics in the initial data, calculating an absorption peak position in the spectrum based on a first-order deviation and a second-order deviation, and the determination and calculation of the spectrum peak position are as follows:

Δxⱼ=xⱼ−xⱼ₋₁

Δ²xⱼ=Δxⱼ−Δxⱼ₋₁

s.t. Δxⱼ=0 and Δ²xⱼ<0

where Δxⱼ and Δ²xⱼ are the first-order deviation and the second-order deviation of a spectrum peak point xⱼ, respectively.

S4, calculating a spectrum peak width based on a half-peak height according to the calculated absorption peak position; the calculation of the spectrum peak width is expressed as follows:

$$\{ \begin{matrix}w = n - m , n > m \\ s . t . {x}_{n} = {x}_{m} = \frac{1}{2} H\end{matrix} ,$$

where n and m are indexes xₙ and xₘ, respectively, and a relative height difference H is expressed as follows:

$$\{ \begin{matrix}H = \vert {x}_{j} - {x}_{i} \vert , {x}_{i} > {x}_{k} \\ H = \vert {x}_{j} - {x}_{k} \vert , {x}_{i} < {x}_{k}\end{matrix} ,$$

where xᵢ and xₖ are a starting point and an ending point of the spectrum peak, respectively.

S5, calculating the symmetric positive semidefinite matrix, a correlation structure matrix and the correlation coefficient of each block according to a sparsity control coefficient of each block; the symmetric positive semidefinite matrix, the correlation structure matrix and the correlation coefficient are expressed as follows:

$${A}_{i} = {s}_{i}^{- 1} ( {q}_{i} {q}_{i}^{T} - {s}_{i} ) {s}_{i}^{- 1}$$

$${γ}_{i} {=}^{△} \frac{1}{{d}_{i}} Tr ( {A}_{i} )$$

$${B}_{i} = \frac{{A}_{i}}{{γ}_{i}} ,$$

where sᵢ=Ωᵢ^{T}D₋ᵢ⁻¹Ωᵢ, qᵢ=Ωᵢ^{T}D₋ᵢ⁻¹y and D₋ᵢ=λI+Σ_{m=1,m≠1}^{g}ΩₘγₘBₘΩₘ^{T}, dᵢ represents a size of the i-th block.

S6, calculating an error value of each block in the initial data based on a cost function, and screening sparse blocks; The error of the cost function is calculated as follows:

L=log|λI+ΩΣ₀Ω^{T}|+y^{T}(λI+ΩΣ₀Ω^{T})⁻¹y,

where Ω∈ [symbol] ^{M×N} is the measuring matrix and Σ₀∈ [symbol] ^{N×N} is the variance matrix of all the blocks, which is expressed as follows:

ΔL(i)=L(Aᵢ^{(t)})−L(Aᵢ^{(t-1)}),

where Aᵢ^{(t)} represents the variance matrix of the i-th block in an iteration of t-th step.

S7, calculating an expectation and a variance of a spectrum posterior probability; the posterior probability expectation is calculated as follows:

$${μ}_{X} = {\sum }_{0} {{Ω}^{T} ( λ I + Ω {\sum }_{0} {Ω}^{T} )}^{- {1}_{y}}$$

$$\sum = ( {\sum }_{0}^{- 1} + \frac{1}{λ} {Ω}^{T} Ω ) ,$$

where y is the compressed matrix of the spectrum obtained by measuring the matrix Ω.

S8, solving superparameters by using a minimization cost function, and updating the noise variance λ in initialization parameters, which is calculated as follows:

$$λ = \frac{1}{M} ( Tr [ \sum {Ω}^{T} Ω ] + {‖ y - \sum μ ‖}_{2}^{2} ) .$$

S9, calculating the relative error of the block correlation coefficient and the current iteration times, if the relative error is less than the set error coefficient η or the current iteration times are larger than the set iteration times T, then turning to S10, otherwise turning to the S5; the relative error judgment conditions are expressed as follows:

$$\frac{‖ {γ}^{( t )} - {γ}^{( t - 1 )} ‖}{‖ {γ}^{( t - 1 )} ‖} \leq η ,$$

where γ^{(t)} is the correlation coefficient of the t-th iteration.

S10, determining a final tea sparse reconstruction data and outputting the data by using the spectrum posterior probability expectation.

Based on the output tea sparse reconstruction data, the sugar content of tea is predicted. Specifically, when Sparse Bayesian learning (SBL) is adopted, the updating spectrum posterior probability expectation formula is expressed as follows:

μ_{w}^{(t+1)}=\hat{α}^{(t)}Σ_{w}^{(t+1)}x,

where \hat{α}^{(t)} represents iterative updating coefficient.

When Block Sparse Bayesian Learning (Block SBL) is adopted, the expectation updating formula is expressed as:

μ_{X}=Σ₀Ω^{T}(λI+ΩΣ₀Ω^{T})⁻¹y,

where y is the compressed matrix of the spectrum obtained by measuring the matrix Ω.

The quantitative analysis index of sugar prediction is the determining coefficient, which is specifically expressed as:

$${R}^{2} = 1 - {\sum }_{i = 1}^{{n}_{0}} ( \frac{{( {z}_{i} - {\hat{z}}_{i} )}^{2}}{{( {z}_{i} - \overline{z} )}^{2}} ) ,$$

where zᵢ represents the real value, \hat{z}ᵢ represents the predicted value, z̅=Σᵢ₌₁ⁿzᵢ/n represents the sample average, nₚ represents the number of samples in the prediction set, and nₚ=47 in this embodiment. By comparing the above method with the commonly used sparse reconstruction method SBL, the method provided by the application may realize the automatic detection and reconstruction of the absorption peak of the near infrared spectrum data of green tea with high precision.

### Embodiment 2

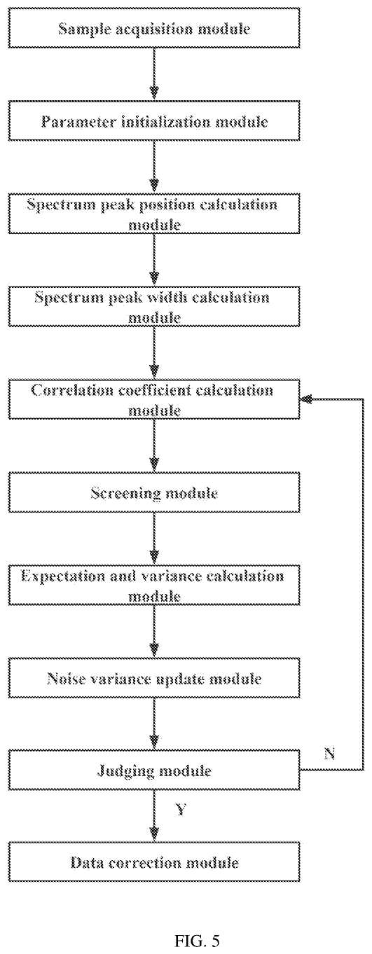

Referring to FIG. 5, this embodiment provides a system for automatically detecting and reconstructing spectrum peaks in near infrared spectrum analysis of tea, including:

a sample acquisition module is used for obtaining the tea samples, obtaining tea near infrared spectrum data and forming the initial data;

a parameter initialization module is used for obtaining the initial data and initializing the parameters of Block Sparse Bayesian Learning method, where the initialization parameter include correlation coefficient γ, iteration times T, noise variance λ, symmetric positive semidefinite matrix A and relative error η of correlation coefficient;

a spectrum peak position calculation module is used for determining the absorption peak position according to the first-order deviation and the second-order deviation of the spectrum data;

a spectrum peak width calculation module is used for determining the peak width according to the half-peak height of the absorption peak;

a correlation coefficient calculation module is used for calculating the sparsity control coefficient of each block to obtain the correlation coefficient;

a screening module is used for calculating the error value of each block and screening the sparse blocks according to the cost function;

an expectation and variance calculation module is used for obtaining the expectation and the variance according to a posterior probability distribution of the spectrum;

a noise variance update module is used for solving the superparameters according to the minimization cost function to obtain noise variance update;

a judging module is used for calculating the relative error of the block correlation coefficient and the current iteration times, and if the relative error is less than the set error coefficient η or the current iteration times are greater than the set iteration times T, the judging is stopped; otherwise, the calculating block correlation coefficient and a block screening module are called again to calculate the sparse reconstruction; and

a data correction module is used for determining and outputting the final tea sparse reconstruction data by using the spectrum posterior probability expectation.

The above is only the preferred embodiment of the application, and it is not used to limit the application. Any modification, equivalent substitution, improvement, etc. made within the spirit and principle of the application should be included in the protection scope of the application.

What is claimed is:

1. A method for automatically detecting and reconstructing spectrum peaks in near infrared spectrum analysis of tea, comprising following steps:
S1, firstly collecting tea samples to be detected, then obtaining near infrared spectrum data of the tea samples, and forming initial data;
S2, firstly obtaining the initial data, and then initializing parameters of Block Sparse Bayesian Learning method comprising a correlation coefficient γ, iteration times T, a noise variance λ, a symmetric positive semidefinite matrix A and a relative error η of the correlation coefficient;
wherein in the S2, an optimization function of the Block Sparse Bayesian Learning method is as follows:
L=log|λI+ΩΣ₀Ω^{T}|+y^{T}(λI+ΩΣ₀Ω^{T})⁻¹y,
wherein I represents an identity matrix, y represents a compressed matrix of spectrum obtained by measuring matrix Ω, Ω∈ [symbol] ^{M×N} is a measuring matrix, and Σ₀∈ [symbol] ^{N×N} is a variance matrix of all blocks expressed as:
Σ₀=diag{γ₁B₁, . . . ,γᵢBᵢ, . . . ,γ_{g}B_{g}},
wherein γᵢ represents a block correlation coefficient of an i-th block, and Bᵢ represents a structure matrix of the i-th block;
S3, calculating an absorption peak position in the spectrum based on a first-order deviation and a second-order deviation and according to spectrum characteristics in the initial data;
S4, calculating a spectrum peak width based on a half-peak height according to the calculated absorption peak position;
S5, calculating the symmetric positive semidefinite matrix, a correlation structure matrix and the correlation coefficient of each block according to a sparsity control coefficient of each block;
S6, calculating an error value of each block in the initial data based on a cost function, and screening sparse blocks;
S7, calculating an expectation and a variance of a spectrum posterior probability;
S8, solving superparameters by using a minimization cost function, and updating the noise variance λ in initialization parameters;
S9, calculating the relative error of the block correlation coefficient and the current iteration times; if the relative error is less than the set error coefficient η or the current iteration times are larger than the set iteration times T, then turning to S10, otherwise turning to the S5; and
S10, determining a final tea sparse reconstruction data and outputting the data by using the spectrum posterior probability expectation.

2. The method for automatically detecting and reconstructing spectrum peaks in near infrared spectrum analysis of tea according to claim 1, wherein in the S3, a spectrum peak position is determined and calculated as follows:
Δxⱼ=xⱼ−xⱼ₋₁
Δ²xⱼ=Δxⱼ−Δxⱼ₋₁
s.t. Δxⱼ=0 and Δ²xⱼ<0
wherein Δxⱼ and Δ²xⱼ are the first-order deviation and the second-order deviation of a spectrum peak point xⱼ, respectively.

3. The method for automatically detecting and reconstructing spectrum peaks in near infrared spectrum analysis of tea according to claim 1, wherein in the S4, the spectrum peak width is calculated as expressed as follows:
$$\{ \begin{matrix}w = n - m , n > m \\ s . t . {x}_{n} = {x}_{m} = \frac{1}{2} H\end{matrix} ,$$
wherein n and m are indexes of xₙ and xₘ respectively, and a relative height difference H is expressed as follows:
$$\{ \begin{matrix}H = \vert {x}_{j} - {x}_{i} \vert , {x}_{i} > {x}_{k} \\ H = \vert {x}_{j} - {x}_{k} \vert , {x}_{i} < {x}_{k}\end{matrix} ,$$
wherein xᵢ and xₖ are a starting point and an ending point of the spectrum peak respectively.

4. The method for automatically detecting and reconstructing spectrum peaks in near infrared spectrum analysis of tea according to claim 1, wherein in the S5, the symmetric positive semidefinite matrix, the correlation structure matrix and the correlation coefficient are expressed as follows:
$${A}_{i} = {s}_{i}^{- 1} ( {q}_{i} {q}_{i}^{T} - {s}_{i} ) {s}_{i}^{- 1}$$
$${γ}_{i} {=}^{△} \frac{1}{{d}_{i}} Tr ( {A}_{i} )$$
$${B}_{i} = \frac{{A}_{i}}{{γ}_{i}} ,$$
wherein sᵢ=Ωᵢ^{T}D₋ᵢ⁻¹Ωᵢ, qᵢ=Ωᵢ^{T}D₋ᵢ⁻¹y and D₋ᵢ=λI+Σ_{m=1,m≠1}^{g}ΩₘγₘBₘΩₘ^{T}, and dᵢ represents a size of the i-th block.

5. The method for automatically detecting and reconstructing spectrum peaks in near infrared spectrum analysis of tea according to claim 1, wherein in the S6, an error of the cost function is calculated as follows:
L=log|λI+ΩΣ₀Ω^{T}|+y^{T}(λI+ΩΣ₀Ω^{T})⁻¹y,
wherein Ω∈ [symbol] ^{M×N} it is the measuring matrix and Σ₀∈ [symbol] ^{N×N} is the variance matrix of all the blocks expressed as follows:
ΔL(i)=L(Aᵢ^{(t)})−L(Aᵢ^{(t-1)}),
wherein Aᵢ^{(t)} represents the variance matrix of the i-th block in an iteration of t-th step.

6. The method for automatically detecting and reconstructing spectrum peaks in near infrared spectrum analysis of tea according to claim 1, wherein in the S7, the posterior probability expectation is calculated as follows:
μ_{X}=Σ₀Ω^{T}(λI+ΩΣ₀Ω^{T})⁻¹y,
wherein y is the compressed matrix of the spectrum obtained by measuring the matrix Ω.

7. The method for automatically detecting and reconstructing spectrum peaks in near infrared spectrum analysis of tea according to claim 1, wherein in the S9, relative error judgment conditions are expressed as follows:
$$\frac{‖ {γ}^{( t )} - {γ}^{( t - 1 )} ‖}{‖ {γ}^{( t - 1 )} ‖} \leq η ,$$
wherein γ^{(t)} is the correlation coefficient of the t-th iteration.

8. A system for automatically detecting and reconstructing spectrum peaks in near infrared spectrum analysis of tea, comprising:
a sample acquisition module used for obtaining tea samples, obtaining tea near infrared spectrum data and forming initial data;
a parameter initialization module used for obtaining the initial data and initializing parameters of Block Sparse Bayesian Learning method, wherein initialization parameters comprise a correlation coefficient γ, iteration times T, a noise variance λ, a symmetric positive semidefinite matrix A and a relative error η of the correlation coefficient;
a spectrum peak position calculation module used for determining the absorption peak position according to the first-order deviation and the second-order deviation of the spectrum data;
a spectrum peak width calculation module used for determining the peak width according to the half-peak height of the absorption peak;
a correlation coefficient calculation module used for calculating the sparsity control coefficient of each block to obtain the correlation coefficient;
a screening module used for calculating the error value of each block and screening the sparse blocks according to the cost function;
an expectation and variance calculation module used for obtaining the expectation and the variance according to a posterior probability distribution of the spectrum;
a noise variance update module used for solving the superparameters according to the minimization cost function to obtain noise variance update;
a judging module used for calculating the relative error of the block correlation coefficient and the current iteration times, wherein if the relative error is less than the set error coefficient η or the current iteration times are greater than the set iteration times T, the judging is stopped; otherwise, the calculating block correlation coefficient and a block screening module are called again to calculate the sparse reconstruction; and
a data correction module used for determining and outputting the final tea sparse reconstruction data by using the spectrum posterior probability expectation.
